(19) United States
(12) Patent Application Publication
(10) Pub. No.: US 2004/0138287 A1
(43) Pub. Date: Jul. 15, 2004

(54) Novel 5-phenyl-1h-indole derivatives as antagonists of interleukine-8 receptors

(76) Inventors: Martine Barth, Asnieres les Dijon (FR); Pierre Dodey, Fontaine Les Dijon (FR); Jean-Luc Paquet, Brognon (FR)

Correspondence Address: MERCHANT & GOULD PC, P.O. BOX 2903, MINNEAPOLIS, MN, 55402-0903, US

(21) Appl. No.: 10/477,638

(22) PCT Filed: May 16, 2002
(86) PCT No.: PCT/FR02/01649

(30) Foreign Application Priority Data
May 17, 2001 (FR) 0106507

(51) Int. Cl. A61K 31/4196; A61K 31/404; C07D 209/18

(52) U.S. Cl. 514/419; 548/494; 514/383

(57) ABSTRACT

The present invention relates to the 5-phenyl-1H-indole derivatives of formula (I):

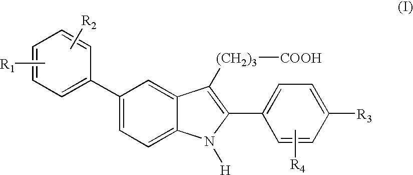

in which R₁, R₂, R₃ and R₄ are as defined in claim 1, and their pharmaceutically acceptable salts, solvates and hydrates.

The invention further relates to pharmaceutical compositions containing them and to their use in the preparation of drugs for the treatment of diseases dependent on the activation of the CXCR2 of interleukin-8 and chemokines of the same family.

The present invention relates to novel 5-phenyl-1H-indole derivatives, to pharmaceutical compositions containing them and to their use in the preparation of drugs for the treatment of diseases dependent on interleukin-8 receptors.

IL-8 (interleukin-8) is a protein of 72 amino acids belonging to the superfamily of proteins capable of attracting leukocytes, said proteins also being referred to as cytokines-CXC or CC intercrine cytokines or, more recently, chemokines (Oppenheim et al., Annu. Rev. Immunol., 1991, 9, 617-648). Different names have been assigned to interleukin-8, such as NAP-1 (neutrophil activating peptide-1), NAF (neutrophil activating factor) and T-cell lymphocyte chemotactic factor. Numerous members of the chemokine family have been described as being involved in inflammatory processes and in leukocyte migration. The chemokine family is made up of two distinct subfamilies: alpha-chemokines and beta-chemokines. Alpha-chemokines, like IL-8, NAP-2 (neutrophil activating peptide-2), MGSA/Gro or Gro-alpha (MGSA=melanoma growth stimulatory activity) and ENA-78 (epithelial cell derived neutrophil activating protein-78), all have effects on the attraction and activation of leukocytes and, more particularly, neutrophils. This subfamily also includes PF-4 (platelet factor-4), beta-thromboglobulin and CTAPIII (connective tissue activating protein-III), which have no effect on neutrophils.

IL-8 was originally identified by its ability to attract and activate polymorphonuclear leukocytes (neutrophils). It has been shown more recently that the expression of IL-8 is rapidly induced in different tissues or cells such as macrophages, fibroblasts, endothelial and epithelial cells and even neutrophils, in response to proinflammatory cytokines such as IL-1-alpha or beta or TNF-alpha (TNF=tumor necrosis factor), or other proinflammatory agents such as LPS (lipopolysaccharide) (Van Damme J., Interleukin-8 and related chemotactic cytokines; 1994; The Cytokines Handbook, 2nd ed., edited by A. W. Thomson, Academic Press, London, pp 185-208). Furthermore, certain literature data have shown high systemic levels of IL-8 in some inflammatory pathological conditions involving neutrophils, suggesting that IL-8 and other chemokines of the same family may be fundamental mediators of neutrophil activation (Van Damme, Interleukin-8 and related chemotactic cytokines; 1994; The Cytokines Handbook, 3rd ed., edited by A. W. Thomson, Academic Press, London, pp 271-311).

Gro-alpha, Gro-beta, Gro-gamma and NAP-2 belong to the chemokine family and, like IL-8, these proteins have also been given different names. Thus Gro-alpha, beta and gamma have respectively been called MGSA-a, b and g (MGSA=melanoma growth stimulatory activity) (Richmond and Thomas, J. Cell Physiol., 1986, 129, 375-384; Cheng et al., J. Immunol., 1992, 148, 451-456). All these chemokines belong to the alpha-chemokine group, which possess an ELR unit (aspartate-leucine-arginate) upstream from the CXC unit characteristic of this subgroup. These chemokines all bind to the type 2 receptor or CXCR2.

Two IL-8 receptors belonging to the family of receptors with seven transmembrane domains coupled to G proteins have been characterized and cloned: the type A IL-8 receptor (IL-8RA), or CXCR1, which binds IL-8 and GCP-2 (granulocyte chemoattractant protein-2) with a high affinity, and the type B IL-8 receptor (IL-8RB), or CXCR2, which has IL-8, GCP-2, Gro-alpha, Gro-beta, Gro-gamma and NAP-2 as specific ligands (Ponath, Exp. Opin. Invest. Drugs, 1998, 7, 1-18). These two receptors exhibit 77% homology in amino acid sequence. Numerous publications have shown abnormally high levels of IL-8 in rheumatoid polyarthritis, septic shock, asthma, cystic fibrosis, myocardial infarction and psoriasis (Baggiolini et al., FEBS Lett., 1992, 307, 97-101; Mille and Krangel, Crit. Rev. Immunol., 1992, 12, 17-46; Oppenheim et al., Annu. Rev. Immunol., 1991, 9, 617-648; Seitz et al., J. Clin. Invest., 1991, 87, 463-469; Miller et al., Am. Rev. Resp. Dis., 1992, 146, 427-432; Donnelly et al., Lancet, 1993, 341, 643-647). IL-8 seems to be involved in pulmonary ischemia/reperfusion phenomena (Sekido et al., Nature, 1993, 365, 654-657). An antibody directed against IL-8 which has the ability to block the IL-8-induced migration of rabbit neutrophils in vitro prevents the tissue damage resulting from a pulmonary ischemia/reperfusion process in the rabbit. IL-8 seems to play a major role in the deterioration due to myocardial hypoxia/reperfusion (Kukielka et al., J. Clin. Invest., 1995, 95, 89-103).

Another study has shown beneficial effects of an IL-8-neutralizing antibody in a model of pleurisy induced by endotoxins in the rabbit (Broadus et al., J. Immunol., 1994, 152, 2960-2967). The involvement of IL-8 in lung inflammations and its deleterious role have been demonstrated with the aid of IL-8-neutralizing antibodies in a model of pulmonary attack induced by an instillation of acid into rabbit lungs (Folkesson et al., J. Clin. Invest., 1995, 96, 107-116) and in a model of acute respiratory distress syndrome induced by endotoxins (Yokoi et al., Lab. Invest., 1997, 76, 375-384). Other reports have shown similar beneficial effects with IL-8-neutralizing antibodies in animal models of dermatosis, arthritis and glomerulonephritis (Akahoshi et al., Lymphokine and Cytokine Res., 1994, 13, 113-116; Nishimura et al., J. Leukoc. Biol., 1997, 62, 444-449; Wada et al., J. Exp. Med., 1994, 180, 1135-1140). Also, mice deficient in interleukin-8 receptors have been produced by removing the gene coding for the murine IL-8 receptor homologous to the human type 2 receptor (CXCR2) (Cacalano et al., Science, 1994, 265, 682-684). Although these mice are healthy, the characteristics of their neutrophils are modified. In fact, their ability to migrate into the peritoneum is reduced in response to an intraperitoneal injection of thioglycolate.

All these results demonstrate that chemokines of the IL-8 family are important mediators of the migration and activation of neutrophils and other cell types, such as endothelial cells, in certain inflammatory conditions. Furthermore, chemokines of the IL-8 family have been described as playing an important role in tumoral growth, the formation of metastases and tumoral angiogenesis in numerous types of cancer (Hebert and Baker, Cancer Invest., 1993, 11, 743-750; Richards et al., Am. J. Surg., 1997, 174, 507-512).

Certain compounds capable of binding to IL-8 receptors are described in the prior art: WO 96/18393, for example, discloses 1-benzyl-2-indolecarboxylic acid derivatives capable of binding to certain IL-8 receptors with an inhibitory effect. More recently, WO 99/06354 has also disclosed compounds derived from urea or thiourea as IL-8 receptor antagonists.

The invention proposes novel non-peptide compounds derived from 5-phenyl-1H-indole which have the property of binding to the CXCR2 of IL-8 and other chemokines of the same family, like NAP-2, Gro-alpha or ENA-78, to behave as antagonists.

The present invention therefore relates to the novel 5-phenyl-1H-indole derivatives of formula (I):

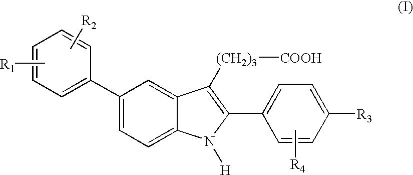

in which:

R₁ is:

a hydrogen atom,

a (C₁-C₄)alkyl group,

a (C₁-C₄)alkoxy group,

a chlorine, bromine or fluorine atom,

a trifluoromethyl group,

a trifluoromethoxy group,

a cyano group,

a nitro group,

an amino group,

a (C₁-C₄)alkenyl group,

a (C₁-C₄)alkylthio group,

a (C₁-C₄)alkanoyl group,

a hydroxy(C₁-C₄)alkyl group,

a group —NH—SO₂—R₅, in which R₅ is a (C₁-C₄)alkyl group,

a trifluoromethanesulfonyl group or

a group —NH—C(O)—R₆, in which R₆ is a hydrogen atom, a

(C₁-C₄)alkyl group or an amino group;

R₂ is a hydrogen atom or a hydroxyl or —NH—C≡N group;

or R₁ and R₂ are bonded to two adjacent carbon atoms of the phenyl group on which they are substituents, forming a triazole group with these two carbon atoms; and

R₃ and R₄ independently of one another are each a hydrogen, chlorine, fluorine or bromine atom or a (C₁-C₄)alkyl or (C₁-C₄)alkoxy group; and their pharmaceutically acceptable salts, solvates and hydrates.

Alkyl is understood as meaning a linear or branched, saturated, monovalent hydrocarbon radical. (C₁-C₄)alkyl is understood as meaning an alkyl radical containing from 1 to 4 carbon atoms.

Alkenyl is understood as meaning a linear or branched, unsaturated, monovalent hydrocarbon radical containing a double bond.

According to another of its features, the invention relates to the compounds of formula (I) in which:

R₁ is:

a hydrogen atom,

a (C₁-C₂)alkyl group,

a methoxy group,

a chlorine, bromine or fluorine atom,

a trifluoromethyl group,

a trifluoromethoxy group,

a cyano group,

a nitro group,

an amino group,

a —CH═CH₂ group,

a methylthio group,

a methanoyl group,

a hydroxymethyl group,

a methanesulfonamido group,

a trifluoromethanesulfonyl group or

a formylamino, acetylamino or (aminocarbonyl)amino group;

R₂ is a hydrogen atom or a hydroxyl or —NH—C≡N group;

or R₁ and R₂ are bonded to two adjacent carbon atoms of the phenyl group on which they are substituents, forming a triazole group with these two carbon atoms; and

R₃ and R₄ independently of one another are each a hydrogen, chlorine or fluorine atom or a methyl group; and their pharmaceutically acceptable salts, solvates and hydrates.

The invention further relates to the compounds of formula (I) in which R₁ and R₂ respectively substitute positions 4 and 3 or, preferably, 3 and 4 of the phenyl to which they are bonded, and their pharmaceutically acceptable salts, solvates and hydrates.

According to another feature, the invention relates to the compounds of formula (I) in which R₂ is a hydroxyl or —NH—C≡N group, and their pharmaceutically acceptable salts, solvates and hydrates.

The compounds of formula (I) in which R₄ substitutes position 3 of the phenyl to which it is bonded, and their pharmaceutically acceptable salts, solvates and hydrates, constitute a further feature of the invention.

The compounds of formula (I) in which R₃ is a chlorine or fluorine atom, preferably fluorine atom, and their pharmaceutically acceptable salts, solvates and hydrates, constitute a further feature of the invention.

The compounds of formula (I) can be salified with a pharmaceutically acceptable mineral or organic base by techniques well known to those skilled in the art. Mineral bases are understood as meaning alkali metal hydroxides such as sodium hydroxide, potassium hydroxide or lithium hydroxide, or alkaline earth metal hydroxides such as calcium hydroxide. Organic bases are understood as meaning primary, secondary or tertiary amines, amino alcohols, certain non-toxic nitrogen heterocycles and basic amino acids. The preferred salts are those of sodium or potassium and those of lysine, arginine or 2-amino-2-methyl-1,3-propanediol.

The compounds of formula (I) according to the invention are prepared e.g. according to SCHEME 1 below, in which R₁ , R₂, R₃ and R₄ are as defined for (I), R is a (C₁-C₄)alkyl group and X and Y independently are a bromine or iodine atom.

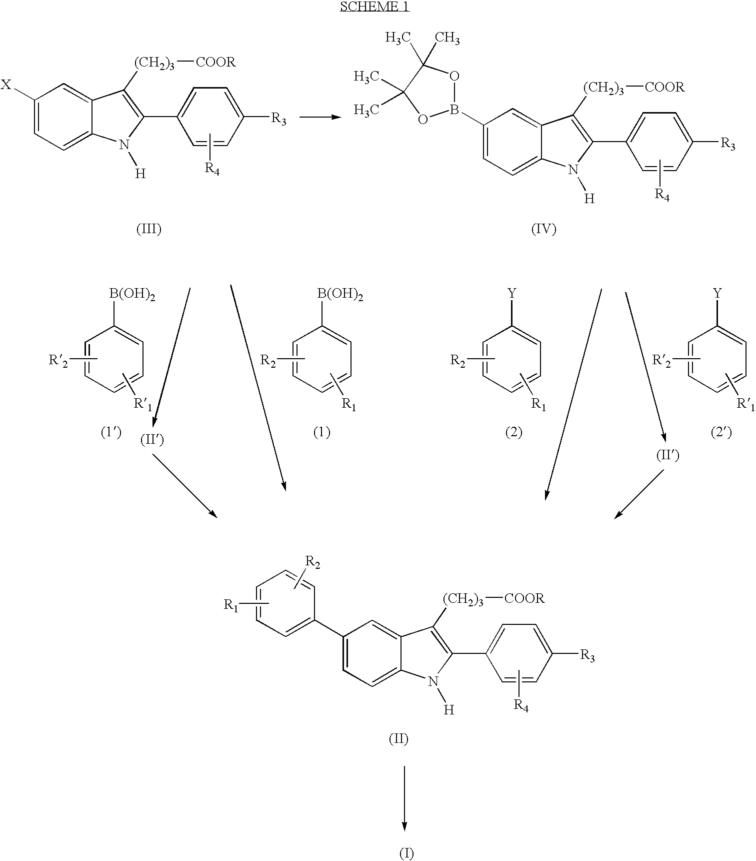

The compounds of formula (I) can be prepared by hydrolysis of the corresponding esters of the formula

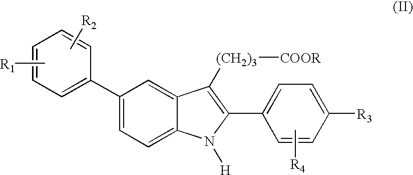

in which R₁ , R₂, R₃ and R₄ are as defined for (I) and R is a (C₁-C₄)alkyl group, particularly an ethyl group or, preferably, a methyl group.

The compounds (II) are novel intermediates and form an integral part of the invention.

The compounds (II) are hydrolyzed to the acids by techniques well known to those skilled in the art, for example by reaction with an aqueous-alcoholic solution of sodium hydroxide.

The compounds of formula (II) can be prepared by means of a Suzuki coupling:

a) either between the compound of formula (III):

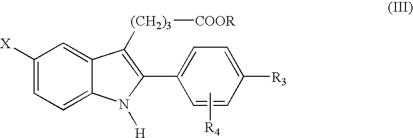

in which R₃ and R₄ are as defined for (I), R is a (C₁-C₄)alkyl group and X is a bromine or iodine atom,

and the boronic acid of formula (1):

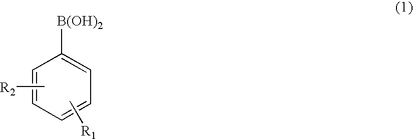

in which R₁ and R₂ are as defined for (I);

b) or between the compound of formula (IV):

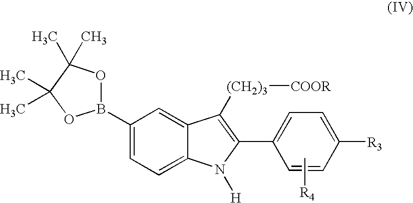

in which R₃ and R₄ are as defined for (I) and R is a (C₁-C₄)alkyl group,

and the compound of formula (2):

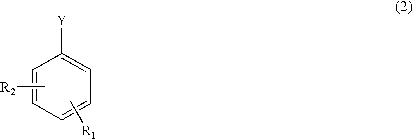

in which R₁ and R₂ are as defined for (I) and Y is a bromine or iodine atom.

This coupling is effected in the presence of a palladium catalyst such as tetrakis(triphenylphosphine)palladium, preferably in the presence of lithium chloride and sodium carbonate.

The coupling described in a) can also be effected between the compound (III) and a boronic acid of formula (1′):

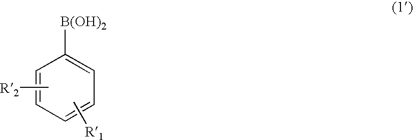

in which R′₁ and R′₂ are respectively R₁ or R₂ or a precursor group or protecting group of the groups R₁ or R₂ that ensures a univocal synthesis of the compounds of formula (I).

Likewise, the coupling described in b) can also be effected between the compound (IV) and a halogenated derivative of formula (2′):

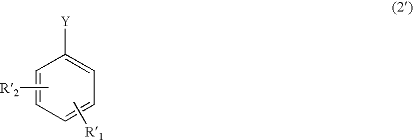

in which R′₁ and R′₂ are as defined for (1′) and Y is a bromine or iodine atom.

According to another alternative, the compounds of formula (II) can be prepared by reacting a tin derivative of formula (3):

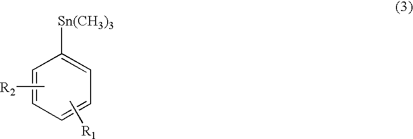

in which R₁ and R₂ are as defined for (I), with the compound of formula (III), in the presence of a palladium catalyst, such as tris(dibenzylidene acetone)dipalladium, and triphenylarsine.

A compound of formula (II) can also be obtained from another compound of formula (II), in one or more steps, by conversion of the substituents R₁ and/or R₂ by conventional methods well known to those skilled in the art.

The boronic acids (1) and (1′) and the halogenated derivatives (2) and (2′) are commercially available compounds or are prepared by techniques well known to those skilled in the art.

The compounds of formula (IV) are prepared e.g. from the compounds of formula (III) by reaction with pinacol borane, in the presence of a palladium catalyst such as 1,1′-bis(diphenylphosphino)ferrocenedichloropalladium(II) and a base such as triethylamine.

The compounds of formula (III) are obtained e.g. by means of a Fischer reaction between the compound of formula (5):

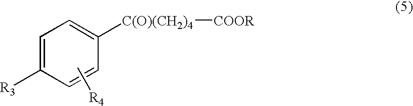

in which R₃ and R₄ are as defined for (I) and R is a (C₁-C₄)alkyl group, and a phenylhydrazine of formula (4):

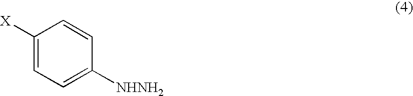

in which X is a bromine or iodine atom.

This Fischer reaction is carried out for example in the presence of zinc dichloride in acetic acid, at a temperature of between 20 and 80° C.

The compounds of formula (4) are commercially available or are obtained by techniques well known to those skilled in the art.

The compounds (5) can be obtained e.g. by means of a Friedel-Crafts reaction between the benzene of formula (6):

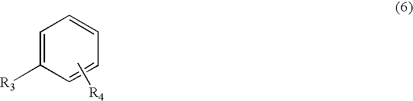

in which R₃ and R₄ are as defined for (I), and the acid chloride Cl—C(O)(CH₂)₄—COOR, in which R is a (C₁-C₄)alkyl group, in the presence of a Lewis acid such as aluminum trichloride.

The compounds of formula (I) according to the invention were subjected to biological studies. Their inhibitory effect on the chemokines IL-8 and Gro-alpha was determined by means of the following in vitro tests:

A) Binding to the IL-8 Receptors

Human IL-8 labeled with iodine-125 ([¹²⁵I]-IL-8) (NEN, Les Ulis) has a specific activity of about 2200 Ci/mmol. Recombinant human CXCR2 was expressed in HEK 293 cells (ATCC, CRL-1573), K-562 cells (ATCC, CCL-243) or THP-1 cells (ATCC, TIB-202). The HEK293 cells are cultured in DMEM (Dulbecco modified Eagle's medium) (GIBCO) containing 4.5 g/l of glucose, 10% of fetal calf serum, 1% of Glutamax, 1% of non-essential amino acids, 1 mM sodium pyruvate, 100 IU/ml of penicillin and 100 μg/ml of streptomycin. The K-562 and THP-1 cells are cultured in RPMI1640 (GIBCO) containing 10% of fetal calf serum, 1% of non-essential amino acids, 1 mM sodium pyruvate, 100 IU/ml of penicillin and 100 μg/ml of streptomycin. The cells are used when the cultures have reached 80% confluence.

The membranes are prepared by the previously described protocol (Bastian et al., Br. J. Pharmacol. 1997, 122, 393-399), except for the homogenization buffer, which was replaced with a buffered saline solution of pH 8.0 containing 20 mM Tris (tris(hydroxymethyl)aminomethane), 1.2 mM MgSO₄ (magnesium sulfate), 0.1 mM EDTA (ethylenediaminetetraacetic acid) and 25 mM NaCl (sodium chloride). The competition experiments are performed in plates of ninety-six 1 ml wells, at room temperature, with a final volume of 0.25 ml. The membranes, diluted in a buffered solution of pH 8.0 containing 20 mM bis-trispropane, 0.4 mM Tris-HCl, 1.2 mM MgSO₄, 0.1 mM EDTA, 25 mM NaCl and 0.03% of CHAPS (3-[(cholamidopropyl)dimethylammonio]-1-propanesulfonate), are incubated with decreasing concentrations of the test compound (from 100 μM to 0.01 nM) and 150 pM [¹²⁵I]-L-8. The non-specific binding is determined in the presence of 300 nM unlabeled IL-8. After 60 minutes of incubation at room temperature, the reaction is stopped by rapid filtration under vacuum on a Whatman GF/C filter previously incubated for 1 hour at +4° C. in a solution containing 1% (weight/volume) of polyethylenimine and 0.5% (weight/volume) of BSA (bovine serum albumin). The filters are washed with a buffered solution of pH 7.4 containing 25 mM NaCl, 1 mM MgSO₄, 0.5 mM EDTA and 10 mM Tris-HCl. The radioactivity retained on the filters is measured in a gamma counter.

The affinities of the compounds described in the present invention were also determined by means of a binding test on whole cells. The transfected THP-1 or K-562 cells are suspended at a rate of 2.5×10⁶ cells/ml in the binding test buffer, namely PBS (phosphate buffered saline) of pH 7.4 containing 0.5% (weight/volume) of BSA but no calcium or magnesium. The competition experiments are performed in plates of ninety-six 1 ml wells with a final volume of 0.25 ml. 0.5×10⁶ cells are incubated with decreasing concentrations of the test compound (100 μM to 0.01 nM) and 150 pM [¹²⁵I]-IL-8. The non-specific binding is determined in the presence of 300 nM non-radiolabeled chemokine. After 90 minutes of incubation at +4° C., the reaction is stopped by rapid filtration under vacuum on a GF/C Whatman filter previously incubated for 1 hour in a 3% (weight/volume) solution of polyethylenimine. The filters are washed with a PBS solution of pH 7.4 containing 0.5 M NaCl. The radioactivity contained in the filters is measured in a gamma counter.

The compounds of formula (I) described in the present invention, tested at a concentration of 10 μM, inhibit the binding of [125I]-IL-8 to CXCR2 by at least 95%.

B) Measurement of the Calcium Flows

The effects of the compounds of the present invention were evaluated on the calcium flows induced by IL-8 or Gro-alpha.

THP-1 cells expressing recombinant CXCR2, U937 cells differentiated with 1% (volume/volume) DMSO (dimethyl sulfoxide) or Eol3 cells are incubated in the presence of the fluorescent indicator Fura-2 AM at a concentration of 5 μM for 1 hour at 37° C. After this loading period, the cells are washed and suspended at a concentration of 1×10⁶ cells/ml in a saline solution of pH 7.4 containing 136 mM NaCl, 4.7 nM KCl, 1.2 mM MgSO₄, 1.6 mM CaCl₂, 1.2 mM KH₂PO₄, 11 mM glucose and 5 mM HEPES (N-[2-hydroxyethyl]piperazine-N′-[2-ethanesulfonic acid]). The cellular suspension (2 ml) is placed in a quartz cell and the fluorescence intensity at 510 nm is measured on an LS50B spectrofluorimeter (Perkin-Elmer) after alternate excitations at 340 nm and 380 nm. The ratio of the fluorescence intensities after excitation at 340 nm and 380 nm is determined and the intracellular calcium concentration [Ca²⁺]i is calculated by the formula

$$[ {Ca}^{2 +} ] i = {K}_{d} \frac{( R - R min )}{( {R}_{max} - R )} ( Sf2 / Sb2 )$$

where:

K_{d} is the affinity constant of the Fura-2/calcium complex, Rmax is the maximum fluorescence intensity determined after the addition of 1 μM of the ionophore Bromo-A23187, Rmin is the minimum ratio determined after the addition of 10 mM EGTA (ethylenebis(oxyethylenenitrilo)tetraacetic acid) following the addition of ionophore, and Sf2/Sb2 is the ratio of the fluorescence values under excitation at 380 nm, determined at Rmin and Rmax respectively.

After a stabilization period of 1 minute, during which the basal intracellular calcium concentration is determined, the test compound or the control vehicle is added to the cells. After an incubation period of 2 minutes, during which the calcium concentration is measured, the cells are stimulated with the different agonists (IL-8 or Gro-alpha). The calcium concentration is measured for 2 minutes.

The compounds of formula (I) described in the present invention inhibit the calcium release induced by IL-8 or Gro-alpha.

The activity of the compounds according to the invention, demonstrated in the biological tests, is indicative of an IL-8-antagonistic action and makes it possible to envisage their use in therapeutics.

Thus the invention further relates to the compounds (I), and their pharmaceutically acceptable salts, solvates and hydrates, for their use as drugs.

Also, according to another of its features, the invention relates to the use of the compounds of formula (I), or one of their pharmaceutically acceptable salts, solvates or hydrates, in the preparation of a drug for the preventive or curative treatment in mammals, especially humans, of diseases dependent on the activation of the CXCR2 of IL-8 and chemokines of the same family, said diseases being generally characterized by a massive invasion of neutrophils.

The following may be mentioned among the diseases which can be treated by the administration of a therapeutically sufficient amount of at least one compound of formula (I): atopic dermatitis, osteoarthritis, rheumatoid arthritis, asthma, chronic pulmonary obstruction, acute respiratory distress syndrome, inflammation of the colon, Crohn's disease, ulcerative colitis, apoplexy attack, myocardial infarction, septic shock, multiple sclerosis, endotoxic shock, psoriasis, septicemia caused by Gram-negative bacteria, toxic shock syndrome, cardiac, pulmonary or renal ischemia/reperfusion phenomena, glomerulonephritis, thrombosis, graft-versus-host reaction, Alzheimer's disease, allograft rejections, malaria, restenosis, angiogenesis, atherosclerosis, osteoporosis, gingivitis, non-physiological release of bone marrow stem cells, diseases caused by respiratory viruses, herpes viruses and hepatitis viruses, meningitis, encephalic herpes, vascularitis of the CNS, cerebral traumatisms, CNS tumors, subarachnoid hemorrhages, post-surgical traumatisms, cystic fibrosis, prenatal labor, cough, pruritus, interstitial pneumonia, hypersensitivity, arthritis induced by crystals, Lyme arthritis, fibrodysplasia ossificans progressiva, acute or chronic pancreatitis, acute alcoholic hepatitis, necrotizing enterocolitis, chronic sinusitis, uveitis, polymyositis, vascularitis, acne, gastric and duodenal ulcers, celiac disease, esophagitis, glossitis, pulmonary obstructions, pulmonary hyperreactivities, bronchiolitis culminating in pneumonia, bronchiectasis, bronchiolitis, proliferative bronchiolitis, chronic bronchitis, dyspnea, emphysema, hypercapnia, hypoxemia, hypoxia, surgical reduction of the pulmonary volume, pulmonary fibrosis, pulmonary hypertension, hypertrophy of the right ventricle, sarcoidosis, attacks of the small bronchioles, ventilation/perfusion errors, respiratory wheezing, lupus, diseases associated with pathological angiogenesis, such as cancer, proliferation of tumor cells and formation of metastasis, for example in the case of melanoma, and cerebral ischemia.

The invention therefore relates to the use of a compound of formula (I), or one of its pharmaceutically acceptable salts, solvates or hydrates, in the preparation of a drug for the treatment of atopic dermatitis, osteoarthritis, rheumatoid arthritis, asthma, chronic pulmonary obstruction, acute respiratory distress syndrome, inflammation of the colon, Crohn's disease, ulcerative colitis, apoplexy attack, myocardial infarction, septic shock, multiple sclerosis, endotoxic shock, psoriasis, septicemia caused by Gram-negative bacteria, toxic shock syndrome, cardiac, pulmonary or renal ischemia/reperfusion phenomena, glomerulonephritis, thrombosis, graft-versus-host reaction, Alzheimer's disease, allograft rejections, malaria, restenosis, angiogenesis, atherosclerosis, osteoporosis, gingivitis, non-physiological release of bone marrow stem cells and diseases caused by respiratory viruses, herpes viruses and hepatitis viruses.

The compounds of formula (I) must be administered in a sufficient amount to antagonize IL-8 by binding competitively to its receptors. The dose of active principle depends on the mode of administration and the type of pathological condition and is generally between 0.01 and 10 mg/kg. The compounds of formula (I) can also be combined with another active principle.

Within the framework of their therapeutic use, the compounds of formula (I) will generally be administered in a variety of forms in combination with the commonly used excipients. Also, the present invention further relates to pharmaceutical compositions containing a compound of formula (I) or one of its pharmaceutically acceptable salts, solvates or hydrates.

The formulation used may be an oral form, for example gelatin capsules, tablets containing the active principle as a solid in a powdered or micronized form, a syrup or a solution containing the active principle in solution, suspension, emulsion or microemulsion.

The formulation may also be presented in a form that can be administered for topical use, for example a cream or lotion or a transdermal device such as an adhesive patch. The active principle may also be formulated for a mode of administration by subcutaneous, intramuscular or intravenous injection.

The PREPARATIONS and EXAMPLES which follow illustrate the invention without however implying a limitation. The following abbreviations are used: s=singlet, m=multiplet, d=doublet, t=triplet, quat=quadruplet, q=quintuplet.

### PREPARATION 1

### Methyl 4-fluoro-ε-oxobenzenehexanoate, Compound 5.1

A suspension of 2.59 g of aluminum chloride in 4 ml of dichloromethane is prepared. It is cooled to −5° C. and a mixture of 0.97 ml of fluorobenzene and 1.31 ml of methyl 6-chloro-6-oxohexanoate in 3 ml of dichloromethane is added gradually, the temperature being kept at between −4 and −7° C. The temperature is then allowed to rise to 20° C. and, after 15 hours, the mixture is hydrolyzed in acidified iced water. It is extracted with dichloromethane and the organic phase obtained is washed with water, dried over magnesium sulfate and concentrated under reduced pressure. 2 g of crude product are recovered in this way and purified by chromatography on silica gel using a petroleum ether/ethyl acetate mixture, 96/4 v/v, as the eluent to give 1.26 g of the expected product in the form of a white powder (yield 63%).

M.p.=58-59° C.

The following compounds are prepared by the same procedure:

Methyl 3,4-difluoro-ε-oxobenzenehexanoate, compound 5.2; m.p.=41-43° C.

Methyl 4-chloro-ε-oxobenzenehexanoate, compound 5.3; m.p.=67-69° C.

Methyl 3,4-dichloro-ε-oxobenzenehexanoate, compound 5.4.

¹H NMR (300 MHz, CDCl₃): 8.01 (d, 1H); 7.77 (dd, 1H); 7.54 (d, 1H); 3.65 (s, 3H); 2.96 (t, 2H); 2.38 (t, 2H); 1.72 (m, 4H).

Methyl 4-chloro-3-methyl-ε-oxobenzenehexanoate, compound 5.5.

Methyl 4-fluoro-3-methyl-ε-oxobenzenehexanoate, compound 5.6.

### PREPARATION 2

### Methyl 5-iodo-2-(4-fluorophenyl)-1H-indole-3-butanoate, Compound III.1

A mixture of 9.79 g of compound 5.1, 14.43 g of 4-iodophenylhydrazine and 8.41 g of zinc chloride in 82 ml of acetic acid is heated at 70° C. for 60 hours. After cooling, 80 ml of water and 100 ml of ethyl acetate are added. After extraction with ethyl acetate, the combined organic phases are washed with water and with saturated aqueous sodium chloride solution and then dried over magnesium sulfate and the solvents are evaporated off under reduced pressure. The residue obtained is purified by chromatography on a silica gel column using a petroleum ether/ethyl acetate mixture, 9/1 v/v, as the eluent.

M.p.=112-114° C.

The following compounds are prepared by the same procedure:

Methyl 5-bromo-2-(4-fluorophenyl)-1H-indole-3-butanoate, compound III.2; m.p.=96-98° C.

Methyl 2-(3,4-difluorophenyl)-5-iodo-1H-indole-3-butanoate, compound III.3; m.p.=118-120° C.

Methyl 2-(4-chlorophenyl)-5-iodo-1H-indole-3-butanoate, compound III.4.

¹H NMR (300 MHz, CDCl₃): 8.03 (s, 1H, NH); 7.94 (s, 1H); 7.46 (m, 5H); 7.15 (d, 1H); 3.62 (s, 3H); 2.83 (t, 2H); 2.34 (t, 2H); 1.95 (q, 2H).

Methyl (3,4-dichlorophenyl)-5-iodo-1H-indole-3-butanoate, compound III.5.

¹H NMR (300 MHz, CDCl₃): 8.02 (s, 1H); 7.95 (s, 1H); 7,62 (d, 1H); 7.55 (d, 1H); 7.47 (dd, 1H); 7.39 (dd, 1H); 7.17 (d, 1H); 3.64 (s, 3H); 2.86 (t, 2H); 2.35 (t, 2H); 1.98 (q, 2H).

Methyl (3,4-difluorophenyl)-5-iodo-1H-indole-3-butanoate, compound III.6; m.p.=143-145° C.

Methyl (4-chloro-3-methylphenyl)-5-iodo-1H-indole-3-butanoate, compound III.7; m.p.=127-128° C.

Methyl (4-fluoro-3-methylphenyl)-5-iodo-1H-indole-3-butanoate, compound III.8; m.p.=119-120° C.

### PREPARATION 3

### Methyl 5-(4-benzyloxyphenyl)-2-(4-fluorophenyl)-1H-indole-3-butanoate, Compound II′.1

0.8 g of compound III.1, 625 mg of 4-benzyloxyphenylboronic acid, 233 mg of lithium chloride, 106 mg of tetrakis(triphenylphosphine)palladium and 4.6 ml of sodium carbonate in 45 ml of methanol and 45 ml of toluene are stirred under reflux for 3 hours 30 minutes. The solvents are evaporated off under reduced pressure and the residue obtained is purified by chromatography on a silica gel column using a petroleum ether/ethyl acetate mixture, 85/15 v/v, as the eluent (yield 57%).

M.p.=113-115° C.

The following compound is prepared by an analogous procedure:

Methyl 2-(4-fluorophenyl)-5-(3,4-diaminophenyl)-1H-indole-3-butanoate, compound II′.2.

¹H NMR (300 MHz, DMSO): 11.10 (s, 1H, NH); 7.65 (dd, 2H); 7.61 (s, 1H); 7.38 (m, 3H); 7.25 (d, 1H); 6.87 (s, 1H); 6.73 (d, 1H); 6.58 (d, 1H); 4.50 (s, 4H); 3.52 (s, 3H); 2.85 (t, 2H); 2.38 (t, 2H); 1.90 (q, 2H).

The compounds (II) shown in TABLE 1 below are also prepared by a procedure analogous to that of PREPARATION 3

TABLE 1

| | |
|---|---|
| | (II) |
| 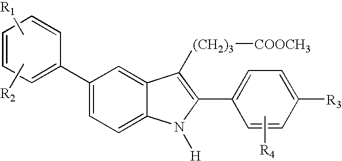 | |

| Compound | R₁ | R₂ | R₃ | R₄ | M.p.: ° C. |
|---|---|---|---|---|---|
| II.1 | 3-CF₃ | H | F | H | 140-142 |
| II.2 | 3-F | H | F | H | (a) |
| II.3 | 4-CH₂CH₃ | H | F | H | 109-111 |
| II.4 | 4-OCF₃ | H | F | H | 145-147 |
| II.5 | 4-Cl | H | F | H | 102-104 |
| II.6 | 4-OCH₃ | H | F | H | 115-117 |
| II.7 | 4-CH₃ | H | F | H | 116-121 |
| II.8 | 4-SCH₃ | H | F | H | (b) |
| II.9 | 4-F | H | F | H | (c) |
| II.10 | 4-CH₂OH | H | F | H | (d) |
| II.11 | 3-Cl | H | F | H | 120-121 |
| II.12 | 4-CH═CH₂ | H | F | H | (e) |
| II.13 | 4-Br | H | F | H | 136-144 |
| II.14 | 3-NHC(O)CH₃ | H | F | H | 172-174 |
| II.15 | 4-C≡N | H | F | H | 163-165 |
| II.16 | 2-F | H | F | H | (f) |
| II.17 | 4-CHO | H | F | H | (g) |
| II.18 | 4-CH₂OH | H | F | 3-F | (h) |
| II.19 | 3-NO₂ | H | F | H | 145-147 |

(a) ¹H NMR(300 MHz, CDCl₃): 8.02(s, 1H, NH); 7.81(s, 1H); 7.53(m, 2H); 7.45(m, 4H); 7.26(m, 1H); 7.18(m, 2H); 7.01(m, 1H); 3.61(s, 3H); 2.95(t, 2H); 2.37(t, 2H); 2.04(q, 2H).
(b) ¹H NMR(300 MHz, CDCl₃): 8.01(s, 1H, NH); 7.79(s, 1H); 7.61(d, 2H); 7.53(m, 2H); 7.43(s, 2H); 7.36(d, 2H); 7.18(m, 2H); 3.62(s, 3H); 2.92(t, 2H); 2.55(s, 3H); 2.46(t, 2H); 2.05(q, 2H).
(c) ¹H NMR(300 MHz, CDCl₃): 8.01(s, 1H, NH); 7.75(d, 1H); 7.62(m, 2H); 7.53(m, 2H); 7.41(s, 2H); 7.19(m, 4H); 3.60(s, 3H); 2.92(t, 2H); 2.35(t, 2H); 2.05(q, 2H).
(d) ¹H NMR(300 MHz, CDCl₃): 8.02(s, 1H, NH); 7.81(s, 1H); 7.68(d, 2H); 7.53(m, 2H); 7.46(m, 4H); 7.19(m, 2H); 4.78(s, 2H); 3.62(s, 3H); 2.93(t, 2H); 2.38(t, 2H); 2.05(q, 2H).
(e) ¹H NMR(300 MHz, CDCl₃): 8.05(s, 1H, NH); 7.82(s, 1H); 7.66(d, 2H); 7.57-7.40(m, 6H); 7.18(m, 2H); 6.78(dd, 1H); 5.80(d, 1H); 5.28(d, 1H); 3.62(s, 3H); 2.90(t, 2H); 2.38(t, 2H); 2.05(q, 2H).
(f) ¹H NMR(300 MHz, CDCl₃): 8.04(s, 1H, NH); 7.78(s, 1H); 7.53(m, 3H); 7.42(s, 2H); 7.20(m, 5H); 3.60(s, 3H); 2.92(t, 2H); 2.35(t, 2H); 2.04(q, 2H).
(g) ¹H NMR(300 MHz, DMSO): 11.38(s, 1H); 10.05(s, 1H, NH); 7.98(m, 5H); 7.68(m, 2H); 7.55(dd, 1H); 7.47(d, 1H); 7.37(t, 2H); 3.55(s, 3H); 2.91(t, 2H); 2.39(t, 2H); 1.91(q, 2H).
(h) ¹H NMR(300 MHz, CDCl₃): 8.02(s, 1H, NH); 7.82(s, 1H); 7.66(m, 2H); 7.51-7.26(m, 6H); 4.77(s, 2H); 3.65(s, 3H); 2.95(t, 2H); 2.38(t, 2H); 2.05(q, 2H).

### PREPARATION 4

### Methyl 2-(4-fluorophenyl)-5-(4-hydroxyphenyl)-1H-indole-3-butanoate, Compound II.20

0.2 g of compound II′.1 solubilized in 8 ml of tetrahydrofuran is stirred under a stream of hydrogen for 48 hours in the presence of palladium-on-charcoal. The reaction mixture is filtered on Célite and the Célite is rinsed with methanol. The filtrate solvents are evaporated off under reduced pressure and the residue obtained is purified by chromatography on a silica gel column using a petroleum ether/ethyl acetate mixture, 8/2 v/v, as the eluent (yield 80%); m.p.=141-143° C.

### PREPARATION 5

### Methyl 2-(4-fluorophenyl)-5-(4-nitrophenyl)-1H-indole-3-butanoate, Compound II.21

255 mg of compound III.1 and 500 mg of trimethyl(4-nitrophenyl)stannane are solubilized in 11.6 ml of dioxane. 73 mg of triphenylarsine and 55 mg of tris(dibenzylidene acetone)dipalladium are added and the reaction mixture is then heated at 50° C. for 24 hours. When the reaction mixture has returned to room temperature, 12.6 ml of water are added. Diethyl ether is added. After filtration and extraction with diethyl ether, the organic phase is washed with water and dried over magnesium sulfate. The solvents are evaporated off under reduced pressure and the residue obtained is purified by chromatography on silica gel using toluene as the eluent (yield 60%); m.p.=190-191° C.

### PREPARATION 6

### Methyl 5-(3-aminophenyl)-2-(4-fluorophenyl)-1H-indole-3-butanoate, Compound II.22

A mixture of 0.76 g of compound II.19 and 2.13 g of tin chloride dihydrate in 30 ml of ethanol is refluxed for 3 hours 20 minutes. After it has returned to room temperature, the reaction mixture is poured onto 65 g of ice, and 4 N aqueous sodium hydroxide solution is added until the pH is 7. After extraction with ethyl acetate and evaporation of the solvents under reduced pressure, the residue obtained is purified by chromatography on a silica gel column using a toluene/ethyl acetate mixture, 85/15 v/v, as the eluent (yield 59%).

¹H NMR (300 MHz, DMSO): 11.20 (s, 1H, NH); 7.70 (s, 1H); 7.65 (dd, 2H); 7.35 (m, 4H); 7.06 (t, 1H); 6.88 (s, 1H); 6.79 (d, 1H); 6.50 (d, 1H); 5.10 (s, 2H, NH₂); 3.51 (s, 3H); 2.90 (t, 2H); 2.38 (t, 2H); 1.90 (q, 2H).

The following compound is prepared in the same manner:

Methyl 5-(4-aminophenyl)-2-(4-fluorophenyl)-1H-indole-3-butanoate, compound II.23; m.p.=65-68° C.

### PREPARATION 7

### Methyl 2-(4-fluorophenyl)-5-(3-methanesulfonamidophenyl)-1H-indole-3-butanoate, Compound II.24

48 μl of mesyl chloride are added to 100 mg of compound II.22 in 1 ml of pyridine. The reaction mixture is heated at 100° C. for 2 hours 15 minutes. After it has returned to room temperature, ice is added and hot 3 N aqueous hydrochloric acid solution is added until the pH is 1. After extraction with ethyl acetate, the organic phase is washed with water and dried over magnesium sulfate and the solvents are then evaporated off under reduced pressure (yield 97%).

¹H NMR (300 MHz, DMSO): 11.30 (s, 1H, NH); 9.80 (s, 1H, NH); 7.79 (s, 1H); 7.67 (dd, 2H); 7.51 (s, 1H); 7.44 (m, 3H); 7.36 (m, 3H); 7.17 (m, 1H); 3.51 (s, 3H); 3.05 (s, 3H); 2.85 (t, 2H); 2.38 (t, 2H); 1.90 (q, 2H).

### PREPARATION 8

### Methyl 2-(4-fluorophenyl)-5-(4,4,5,5-tetramethyl-1,3,2-dioxaborolan-2-yl)-1H-indole-3-butanoate, Compound IV.1

4.1 g of compound III.1, 3.9 ml of triethylamine and 2.1 ml of pinacol borane are added successively to 40 ml of dioxane in the presence of 230 mg of 1,1′-bis(diphenylphosphino)ferrocenedichloropalladium(II). The reaction mixture is heated at 80° C. for 2 hours. The suspension is then filtered and washed with toluene, after which the filtrate is extracted with toluene and the organic phase is washed with water and dried over magnesium sulfate (yield 98%); m.p.=166-168° C.

The following compounds are prepared by an analogous procedure:

Methyl 2-(4-chlorophenyl)-5-(4,4,5,5-tetramethyl-1,3,2-dioxaborolan-2-yl)-1H-indole-3-butanoate, compound IV.2; m.p.=164-166° C.

Methyl 2-(3,4-dichlorophenyl)-5-(4,4,5,5-tetramethyl-1,3,2-dioxaborolan-2-yl)-1H-indole-3-butanoate, compound IV.3.

¹H NMR (300 MHz, CDCl₃): 8.04 (s, 1H); 7.98 (s, 1H, NH); 7.64 (dd, 1H); 7.57 (d, 1H); 7.47 (d, 1H); 7.34 (dd, 1H); 7.29 (d, 1H); 3.55 (s, 3H); 2.86 (t, 2H); 2.28 (t, 2H); 1.97 (q, 2H).

Methyl 2-(3,4-difluorophenyl)-5-(4,4,5,5-tetramethyl-1,3,2-dioxaborolan-2-yl)-1H-indole-3-butanoate, compound IV.4; m.p.=175-177° C.

Methyl 2-(4-chloro-3-methylphenyl)-5-(4,4,5,5-tetramethyl-1,3,2-dioxaborolan-2-yl)-1H-indole-3-butanoate, compound IV.5; m.p.=164-165° C.

Methyl 2-(4-fluoro-3-methylphenyl)-5-(4,4,5,5-tetramethyl-1,3,2-dioxaborolan-2-yl)-1H-indole-3-butanoate, compound IV.6; m.p.=150-152° C.

### PREPARATION 9

### 4-Bromophenyl trifluoromethyl sulfoxide, Compound 2.1

358 mg of hydrogen peroxide and 360 μl of acetic acid are added at 40° C. to 474 mg of 1-bromo-4-[(trifluoromethyl)thio]benzene in 2.55 ml of acetic acid. 322 mg of hydrogen peroxide are then added. The reaction mixture is refluxed for 3 hours 25 minutes and . . . at room temperature for 12 hours and is then poured onto 9 ml of ice. The white precipitate obtained is filtered off, washed with cold water and then purified by chromatography on a silica gel column using a petroleum ether/ethyl acetate mixture, 9/1 v/v, as the eluent (yield 53%).

M.p.=64-66° C.

### PREPARATION 10

### N-(2-iodophenyl)cyanamide, Compound 2.2

1.05 g of cyanogen bromide in 7 ml of diethyl ether are added dropwise at a temperature below 0° C., under an inert atmosphere, to a solution of 3.22 g of 3-iodoaniline in 13 ml of diethyl ether. The reaction mixture is stirred for 20 minutes at this temperature and then for 17 hours at room temperature. The suspension is filtered and washed with diethyl ether. The filtrate solvents are evaporated off under reduced pressure and the residue obtained is purified by chromatography on a silica gel column using a toluene/ethyl acetate mixture, 95/5 v/v, as the eluent; m.p.=117-118° C.

### PREPARATION 11

### Methyl 2-(4-fluorophenyl)-5-(3-fluoro-4-hydroxyphenyl)-1H-indole-3-butanoate, Compound II.25

Compound II.25 is prepared from compound IV.1 and 4-bromo-2-fluorophenol by a procedure analogous to that of PREPARATION 3.

¹H NMR (300 MHz, DMSO): 11.25 (s, 1H, NH); 7.77 (s, 1H); 7.66 (m, 2H); 7.46 (dd, 1H); 7.37 (m, 5H); 7.01 (t, 1H); 3.55 (s, 3H); 2.87 (t, 2H); 2.39 (t, 2H); 1.90 (q, 2H).

The compounds (II) shown in TABLES 2 and 3 below are prepared by an analogous procedure.

TABLE 2

| | |
|---|---|
| | (II) |
| 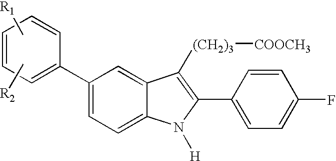 | |

| Compound | —R₁ | —R₂ | M.p.; ° C., or ¹H NMR |
|---|---|---|---|
| II.26 | 3-CH₃ | 4-OH | (b₁) |
| II.27 | H | 3-OH | (c₁) |
| II.28 | 3-SO₂CF₃ | H | 158-160 |
| II.29 | 3-NHCHO^{(a1)} | H | (d₁) |
| II.30 | 3-NO₂ | 4-OH | (e₁) |
| II.31 | H | 3-NH—C≡N | (f₁) |

^{(a1)}from the iodinated derivative described in J.A.C., 1997, 119, 7271-7280
(b₁) ¹H NMR(300 MHz, CDCl₃): 7.94(s, 1H, NH); 7.64(s, 1H); 7.46(m, 2H); 7.32(m, 4H); 7.09(m, 2H); 6.79(d, 1H); 3.52(s, 3H); 2.87(t, 2H); 2.28(m, 5H); 1.97(q, 2H).
(c₁) ¹H NMR(300 MHz, DMSO): 11.25(s, 1H, NH); 9.45(s, 1H, OH); 7.77(s, 1H); 7.67(m, 2H); 7.38(m, 4H); 7.24(t, 1H); 7.10(m, 2H); 6.71(d, 1H); 3.56(s, 3H); 2.88(t, 2H); 2.39(t, 2H); 1.91(q, 2H).
(d₁) ¹H NMR(300 MHz, DMSO): 11.30(s, 1H, NH); 10.27(s, 1H, NH); 8.32(s, 1H); 7.87(s, 1H); 7.78(s, 1H); 7.68(m, 2H); 7.57(m, 1H); 7.40(m, 6H); 3.55(s, 3H); 2.89(t, 2H); 2.39(t, 2H); 1.90(q, 2H).
(e₁) ¹H NMR(300 MHz, DMSO): 11.30(s, 1H); 8.15(d, 1H); 7.95(m, 2H); 7.68(m, 2H); 7.45(m, 4H); 7.22(d, 1H); 3.60(s, 3H); 2.88(t, 2H); 2.42(t, 2H); 1.92(q, 2H).
(f₁) ¹H NMR(300 MHz, DMSO): 11.30(s, 1H, NH); 7.80(s, 1H); 7.68(m, 2H); 7.45(d, 2H); 7.38(m, 5H); 7.20(m, 1H); 6.92(dd, 1H); 3.55(s, 3H); 2.89(t, 2H); 2.39(t, 2H); 1.88(q, 2H).

TABLE 3

| | |
|---|---|
| | (II) |
| 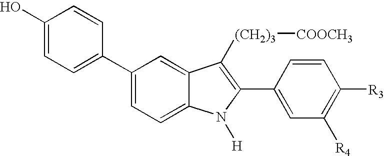 | |

| Compound | —R₃ | —R₄ | M.p.; ° C. |
|---|---|---|---|
| II.32 | Cl | H | |
| II.33 | Cl | Cl | (a₂) |
| II.34 | F | F | (b₂) |
| II.35 | Cl | —CH₃ | 164-165 |
| II.36 | F | —CH₃ | 160-161 |

(a₂) ¹H NMR(300 MHz, DMSO): 11.30(s, 1H, NH); 9.50(s, 1H, OH); 7.87(d, 1H); 7.76(d, 2H); 7.64(dd, 1H); 7.50(d, 2H); 7.37(m, 2H); 6.85(d, 2H); 3.55(s, 3H); 2.92(t, 2H); 2.39(t, 2H); 1.90(q, 2H).
(b₂) ¹H NMR(300 MHz, DMSO): 11.25(s, 1H, NH); 9.48(s, 1H, OH); 7.75(s, 1H); 7.70-7.45(m, 5H); 7.37(m, 2H); 7.86(d, 2H); 3.58(s, 3H); 2.90(t, 2H); 2.40(t, 2H); 1.90(q, 2H).

### PREPARATION 12

### Methyl 5-(1H-1,2,3-benzotriazol-5-yl)-2-(4-fluorophenyl)-1H-indole-3-butanoate, Compound II.37

36 mg of compound II′.2 in 2 ml of a 50% ethanol/water solution are acidified to pH 2 by the addition of 2 N aqueous hydrochloric acid solution. 7 mg of sodium nitrite in a minimum amount of water are then added dropwise at 0° C. The reaction mixture is stirred for 10 minutes at 0° C. and then for 3 hours 45 minutes at room temperature. After the addition of water and ethyl acetate and a few minutes' stirring, the organic phase is separated from the aqueous phase. The aqueous phase is extracted with ethyl acetate. The combined organic phases are washed with water and dried over magnesium sulfate. After evaporation of the solvents under reduced pressure, the residue obtained is purified by chromatography on a silica gel column using a dichloromethane/ethyl acetate mixture, 8/2 v/v, as the eluent (yield 27%).

¹H NMR (300 MHz, CDCl₃): 8.45 (s, 1H, NH); 8.05 (s, 1H); 7.93 (d, 1H); 7.82 (s, 1H); 7.72 (d, 1H); 7.50 (m, 2H); 7.39 (s, 2H); 7.12 (t, 2H); 3.60 (s, 3H); 2.95 (t, 2H); 2.36 (t, 2H); 2.05 (q, 2H).

### PREPARATION 13

### Methyl 2-(4-chlorophenyl)-5-[3-(cyanoamino)phenyl]-1H-indole-3-butanoate, Compound II.38

By following a procedure analogous to Preparation 11 starting from compound IV-2 and N-(3-iodophenyl)cyanamide, the expected product is obtained in the form of a beige solid (yield 16%).

NMR (300 MHz, DMSO) δ: 11.35 (s, 1H); 7.81 (s, 1H); 7.57 (d, 2H); 7.58 (d, 2H); 7.39 (m, 4H); 7.19 (s, 1H); 6.91 (d, 1H); 3.55 (s, 3H); 2.90 (m, 2H); 2.39 (m, 2H); 1.90 (m, 2H).

### EXAMPLE 1

### 2-(4-Fluorophenyl)-5-(4-hydroxyphenyl)-1H-indole-3-butanoic acid

A mixture of 130 mg of compound II.20, 0.65 ml of 1 N aqueous sodium hydroxide solution and 2 ml of dioxane is prepared. The reaction mixture is refluxed for 2 hours 15 minutes. The solvents are evaporated off under reduced pressure and the residue is taken up with water and then acidified to pH 1 with 2 N aqueous hydrochloric acid solution. The precipitate obtained is filtered off, washed with water and petroleum ether and then dried under reduced pressure (yield 80%); m.p.=180-183° C.

EXAMPLES 2 to 34 shown in TABLE 4 below are prepared by an analogous procedure.

TABLE 4

| | |
|---|---|
| | (I) |
| 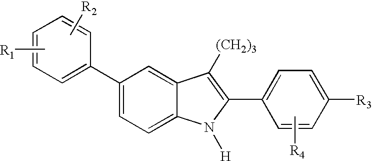 | |

| EX- | | | | | |
|---|---|---|---|---|---|
| AMPLE | —R₁ | —R₂ | —R₃ | —R₄ | M.p.(° C.) |
| 2 | 3-CF₃ | H | F | H | 171-173 |
| 3 | 3-F | H | F | H | 147-149 |
| 4 | 4-CH₂CH₃ | H | F | H | 157-159 |
| 5 | 4-OCF₃ | H | F | H | 173-175 |
| 6 | 4-Cl | H | F | H | 191-193 |
| 7 | 4-OCH₃ | H | F | H | 193-195 |
| 8 | 4-CH₃ | H | F | H | 185-189 |
| 9 | 4-SCH₃ | H | F | H | 145-150 |
| 10 | 4-F | H | F | H | 186-188 |
| 11 | 4-CH₂OH | H | F | H | 148-150 |
| 12 | 3-Cl | H | F | H | 88-90 |
| 13 | 4-CH═CH₂ | H | F | H | (a₃) |
| 14 | 4-Br | H | F | H | 183-185 |
| 15 | 4-NHCOCH₃ | H | F | H | 145-147 |
| 16 | 4-C≡N | H | F | H | 232-235 |
| 17 | 2-F | H | F | H | 174-176 |
| 18 | 4-CHO | H | F | H | 224-225 |
| 19 | 3-NHSO₂CH₃ | H | F | H | 220-222 |
| 20 | 4-NH₂ | H | F | H | 260 |
| 21 | 4-CH₂OH | H | F | 3-F | 148-150 |
| 22 | 3-F | 4-OH | F | H | 82-85 |
| 23 | 3-CH₃ | 4-OH | F | H | 89-90 |
| 24 | H | 3-OH | F | H | 191-193 |
| 25 | 3-SO₂CF₃ | H | F | H | 410 |
| 26 | 3-NHCHO | H | F | H | 219-221 |
| 27 | 3-NO₂ | 4-OH | F | H | 83-85 |
| 28 | 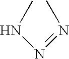 | | F | H | 135 |
| 29 | H | 3-NH—C≡N | F | H | 230 |
| 30 | H | 4-OH | Cl | H | 208-210 |
| 31 | H | 4-OH | Cl | 3-Cl | 198-200 |
| 32 | H | 4-OH | F | 3-F | 87-90 |
| 33 | H | 4-OH | Cl | 3-CH₃ | 198-200 |
| 34 | H | 4-OH | F | 3-CH₃ | 168-170 |

(a₃) ¹H NMR(300 MHz, DMSO): 12.05(s, 1H); 11.25(s, 1H); 7.89(s, 1H); 7.70(m, 4H); 7.56(d, 2H); 7.44(s, 2H); 7.36(m, 2H); 6.80(dd, 1H); 5.87(d, 1H); 5.28(d, 1H); 2.90(t, 2H); 2.32(t, 2H); 1.92(q, 2H).

### EXAMPLE 35

### 5-[3-[(Aminocarbonyl)amino]phenyl]-2-(4-chlorophenyl)-1H-indole-3-butanoic acid

a) 2-(4-chlorophenyl)-5-[3-(cyanoamino)phenyl]-1H-indole-3-butanoic acid

By following a procedure analogous to Example 1 starting from compound II.38, the expected acid is obtained with a yield of 55%.

b) 5-[3-[(aminocarbonyl)amino]phenyl]-2-(4-chlorophenyl)-1H-indole-3-butanoic acid

The compound obtained in step a) above (111 mg, 0.26 mmol) is suspended in 6 ml of 1 N hydrochloric acid and the mixture is refluxed gently for 10 min. After cooling, the reaction medium is diluted with 10 ml of water and then extracted with ethyl acetate. The organic phase is washed with water and then dried and concentrated under reduced pressure. The residue is crystallized from 3 ml of dichloromethane, filtered off and purified by chromatography on silica gel using a dichloromethane/methanol mixture, 9/1 v/v, as the eluent to give the expected acid in the form of a beige solid (yield 9%).

NMR (300 MHz, DMSO) δ: 12.12 (s, 1H); 11.32 (s, 1H); 8.67 (s, 1H); 7.81 (s, 1H); 7.70 (m, 3H); 7.58 (d, 2H); 7.40 (m, 3H); 7.29 (t, 1H); 7.21 (d, 1H); 5.91 (s, 2H); 2.89 (m, 2H); 2.29 (m, 2H); 1.88 (m, 2H).

1. Compounds of formula (I): 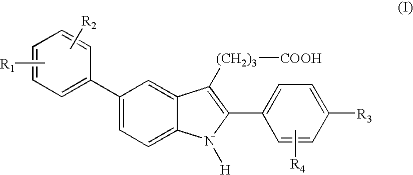
in which:
R₁ is:
a hydrogen atom,
a (C₁-C₄)alkyl group,
a (C₁-C₄)alkoxy group,
a chlorine, bromine or fluorine atom,
a trifluoromethyl group,
a trifluoromethoxy group,
a cyano group,
a nitro group,
an amino group,
a (C₁-C₄)alkenyl group,
a (C₁-C₄)alkylthio group,
a (C₁-C₄)alkanoyl group,
a hydroxy(C₁-C₄)alkyl group,
a group —NH—SO₂—R₅, in which R₅ is a (C₁-C₄)alkyl group,
a trifluoromethanesulfonyl group or
a group —NH—C(O)—R₆, in which R₆ is a hydrogen atom, a (C₁-C₄)alkyl group or an amino group;
R₂ is a hydrogen atom or a hydroxyl or —NH—C≡N group;
or R₁ and R₂ are bonded to two adjacent carbon atoms of the phenyl group on which they are substituents, forming a triazole group with these two carbon atoms; and
R₃ and R₄ independently of one another are each a hydrogen, chlorine, fluorine or bromine atom or a (C₁-C₄)alkyl or (C₁-C₄)alkoxy group;
and their pharmaceutically acceptable salts, solvates and hydrates.

2. Compounds according to claim 1, characterized in that:
R₁ is:
a hydrogen atom,
a (C₁-C₂)alkyl group,
a methoxy group,
a chlorine, bromine or fluorine atom,
a trifluoromethyl group,
a trifluoromethoxy group,
a cyano group,
a nitro group,
an amino group,
a —CH═CH₂ group,
a methylthio group,
a methanoyl group,
a hydroxymethyl group,
a methanesulfonamido group,
a trifluoromethanesulfonyl group or
a formylamino, acetylamino or (aminocarbonyl)amino group;
R₂ is a hydrogen atom or a hydroxyl or —NH—C≡N group;
or R₁ and R₂ are bonded to two adjacent carbon atoms of the phenyl group on which they are substituents, forming a triazole group with these two carbon atoms; and
R₃ and R₄ independently of one another are each a hydrogen, chlorine or fluorine atom or a methyl group.

3. Compounds according to claim 1 or 2, characterized in that R₁ and R₂ respectively substitute positions 4 and 3, or 3 and 4, of the phenyl to which they are bonded.

4. Compounds according to claim 3, characterized in that R₁ and R₂ respectively substitute positions 3 and 4 of the phenyl to which they are bonded.

5. Compounds according to claim 4, characterized in that R₂ is a hydroxyl or —NH—C≡N group.

6. Compounds according to any one of claims 1 to 5, characterized in that R₄ substitutes position 3 of the phenyl to which it is bonded.

7. Compounds according to claim 6, characterized in that R₃ is a chlorine or fluorine atom.

8. Compounds according to claim 7, characterized in that R₃ is a fluorine atom.

9. Esters of formula (II): 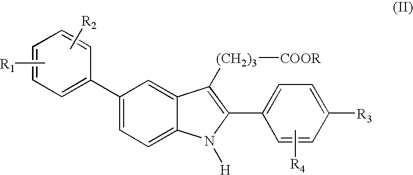
in which R₁ , R₂, R₃ and R₄ are as defined for (I) and R is a (C₁-C₄)alkyl group.

10. Compound according to any one of claims 1 to 8 for its use as a drug.

11. Pharmaceutical composition containing a compound according to any one of claims 1 to 8.

12. Use of a compound according to any one of claims 1 to 8 in the preparation of a drug for the treatment of diseases dependent on the activation of the CXCR2 of interleukin-8 and chemokines of the same family.

13. Use according to claim 12 in the preparation of a drug for the treatment of atopic dermatitis, osteoarthritis, rheumatoid arthritis, asthma, chronic pulmonary obstruction, acute respiratory distress syndrome, inflammation of the colon, Crohn's disease, ulcerative colitis, apoplexy attack, myocardial infarction, septic shock, multiple sclerosis, endotoxic shock, psoriasis, septicemia caused by Gram-negative bacteria, toxic shock syndrome, cardiac, pulmonary or renal ischemia/reperfusion phenomena, glomerulonephritis, thrombosis, graft-versus-host reaction, Alzheimer's disease, allograft rejections, malaria, restenosis, angiogenesis, atherosclerosis, osteoporosis, gingivitis, non-physiological release of bone marrow stem cells and diseases caused by respiratory viruses, herpes viruses and hepatitis viruses.
